(19) United States
(12) Patent Application Publication
(10) Pub. No.: US 2021/0007894 A1
(43) Pub. Date: Jan. 14, 2021

(54) Ski Goggles Convenient to Replace

(71) Applicant: Zhu Yan, Guangzhou (CN)

(72) Inventor: Zhu Yan, Guangzhou (CN)

(21) Appl. No.: 16/568,296

(22) Filed: Sep. 12, 2019

(30) Foreign Application Priority Data
Jul. 10, 2019 (CN) 2019210884420

(51) Int. Cl. A61F 9/02; A42B 3/18; G02C 7/10

(52) U.S. Cl. CPC A61F 9/026; G02C 7/10; A42B 3/18

(57) ABSTRACT

Ski goggles convenient to replace comprise a ski goggles body, a mounting pad and a face sponge; a groove is formed in the inner side of the ski goggles body, the mounting pad is mounted on the groove, the groove is provided with two or more through holes, the mounting pad is provided with columns with the same number as the through holes, a limiting device in limiting fit with the corresponding through hole is formed on each column, each limiting device is positioned on one side of the corresponding column and protrudes out of the side wall of the column, the limiting devices protrude away from the center of the ski goggles body, the limiting device positioned on the nose part protrudes towards the center of the ski goggles body, and the characteristic of facilitating mounting and replacing of the face sponge is realized.

### TECHNICAL FIELD

The present utility model relates to ski goggles, in particular to ski goggles convenient to replace.

### BACKGROUND ART

Ski goggles are glasses used for protecting eyes of a user during skiing, a face sponge is arranged at the position, attached to the face, of the rear portion of the ski goggles, and the face sponge has the functions of enabling the face to fit the ski goggles better, reducing gaps between the face and the ski goggles, and preventing snowflakes from entering the ski goggles to influence eyeline. Owing to the face sponge, the skiing experience of people is greatly improved, and the face sponge is welcomed by the markets. However, the face sponge of existing ski goggles is pasted with a body, and cannot be replaced, once the face sponge is dirty, the ski goggles will be eliminated together with the face sponge, and resource waste may be caused. For example, the Chinese patent application No. 201621252022.8 with the announcement date of Jul. 14, 2017 discloses detachable face sponge type ski goggles which solve the problem of resource waste caused by the fact that the face sponge cannot be replaced in the prior art. The ski goggles in the present utility model includes a ski goggles body and a face sponge, and further includes a detachable rack, wherein the detachable rack is arranged between the ski goggles body and the face sponge, the detachable rack is tightly fixed to the ski goggles body through a fastener, the fastener is arranged on the detachable rack and/or the ski goggles body, and the face sponge is tightly attached to the detachable rack. The skin goggles in the present utility model are simple in structure and convenient to operate, resource waste of ski goggles caused by replacement of the face sponge is greatly reduced, and the market prospect is great. According to the present utility model, the detachable rack is provided with a clamping hook at the edge, and the side edge of the ski goggles body is provided with a clamping groove so as to fix the detachable rack, due to the fact that the edge of the ski goggles body is provided with the clamping groove, the appearance of the ski goggles may be influenced, in addition, the side edge of the ski goggles body is provided with the clamping groove, the detachable rack is provided with the clamping hook, the clamping hook is clamped into the clamping groove from the side edge, and during disassembling, the clamping hook needs to be separated from the clamping groove, so that difficulty in disassembling when accessories are replaced is easily caused; and meanwhile, the ski goggles are positioned on a nose part, and the circumstance that the fastener falls off easily due to upward pressing of the nose on the ski goggles during skiing is not considered.

### SUMMARY OF THE UTILITY MODEL

In order to solve the problems in the prior art, the present utility model provides ski goggles convenient to replace, and the characteristics that a mounting pad is convenient to disassemble and is good in mounting stability are realized by changing a limiting fit device structure of the mounting pad and a ski goggles body.

In order to achieve the above purpose, the technical scheme of the present utility model is as follows: ski goggles convenient to replace are provided and includes a ski goggles body, a mounting pad and a face sponge, a groove is formed in the inner side of the ski goggles body, the mounting pad is mounted on the groove, the groove is provided with two or more through holes, the mounting pad is provided with columns with the same number as the through holes, each column is provided with a limiting device in limiting fit with the corresponding through hole, each limiting device is positioned on one side of the corresponding column and protrudes out of the side wall of the column, each limiting device protrudes away from the center of the ski goggles body, and the limiting device positioned on the nose part protrudes towards the center of the ski goggles body.

By the arrangement, when the face sponge is replaced, the mounting pad is pressed with fingers to apply a force towards the palm, each limiting device moves into the range of the corresponding through hole, then the mounting pad is pulled outwards, the mounting pad moves outwards to release limiting fit, and replacement is facilitated; each limiting device is positioned on one side of the corresponding column and protrudes of the side wall of the corresponding column, thus, each limiting device is only in contact with one side of the corresponding through hole, a buffer space is formed between each column and the other side of the corresponding through hole, so that assembling and disassembling of each limiting device are facilitated, and the size is small; when the ski goggles are used, the protruded portion of the limiting device positioned on the nose part faces the inside of the ski goggles body, the nose of a human body provides a lifting force for the ski goggles, the limiting device positioned on the nose part tends to move towards the inside of the ski goggles body, then limiting fit is more fastened when the ski goggles are used, and the mounting stability of the mounting pad is improved.

Further, the upper end face of the side wall of the groove is in chamfering transition arrangement, so that disassembling and assembling of the mounting pad are guided favorably, and the face sponge can be conveniently replaced.

Further, a first inner cavity is formed in the ski goggles body, a goggles lens, a sealing ring and an anti-ultraviolet lens are arranged in the first inner cavity from inside to outside, the sealing ring is positioned between the goggles lens and the anti-ultraviolet lens, by placing the goggles lens and the anti-ultraviolet lens in the first inner cavity, accessories are prevented from falling off, meanwhile, the sealing ring is arranged between the goggles lens and the anti-ultraviolet lens, thus, the sealing performance is enhanced while friction generated between the goggles lens and the anti-ultraviolet lens is prevented.

Further, a second inner cavity is formed between the first inner cavity and the groove in the ski goggles body, a filling sponge is arranged in the second inner cavity, and is closely attached to the inner wall of the second inner cavity, and the filling sponge stops solid matters such as snowflakes from splashing into the ski goggles.

Further, the mounting pad positioned on the nose part is provided with two or more notches, due to the fact that the notches are formed in the nose part, a deformation space is provided for the mounting pad by the notches, the mounting pad fits the structure of the nose better, the contact area with the nose is increased, then the friction force of the contact surface is further increased, a buffer space is provided for deformation caused by the lifting force on the ski goggles, the ski goggles conform to the structure of the human body, wearing comfort degree is improved, and the ski goggles are not easy to fall off.

Further, limiting protruding devices protruding towards the center of the first inner cavity are respectively arranged on the inner walls, located at relative positions of the nose part and located at two sides of the nose part, of the first inner cavity, so that accessories such as the goggles lens are more stably fixed in the first inner cavity, and further limiting is implemented to prevent the accessories from falling off.

Further, the face sponge is arranged on the mounting pad, and is bonded with the mounting pad, so that the mounting firmness of the face sponge is ensured.

Further, the outer wall of the ski goggles body is provided with a breathable through groove, by the arrangement, gas exchange with the outside is implemented in cooperation with the breathable through groove, and breathability inside the ski goggles body is improved.

### BRIEF DESCRIPTION OF THE DRAWINGS

FIG. 1 is a schematic structural exploded view of an embodiment of the present utility model;

FIG. 2 is a schematic structure diagram of a ski goggles body of an embodiment of the present utility model;

FIG. 3 is a schematic structure diagram of a mounting pad of an embodiment of the present utility model;

FIG. 4 is a schematic cross-sectional view taken along the direction A-A of FIG. 3; and

FIG. 5 is a front view of a ski goggles body of an embodiment of the present utility model.

### DESCRIPTION OF REFERENCE NUMERALS

1—ski goggles body, 2—mounting pad, 3—face sponge, 4—groove, 5—through hole, 6—column, 7—limiting device, 8—chamfering transition arrangement, 9—first inner cavity, 10—goggles lens, 11—sealing ring, 12—anti—ultraviolet lens, 13—filling sponge, 14—breathable through groove, 15—notch, 16—limiting protruding device, 17—second inner cavity.

### DETAILED DESCRIPTION OF THE UTILITY MODEL

The present utility model is further described in detail below with reference to the accompanying drawings and specific embodiments.

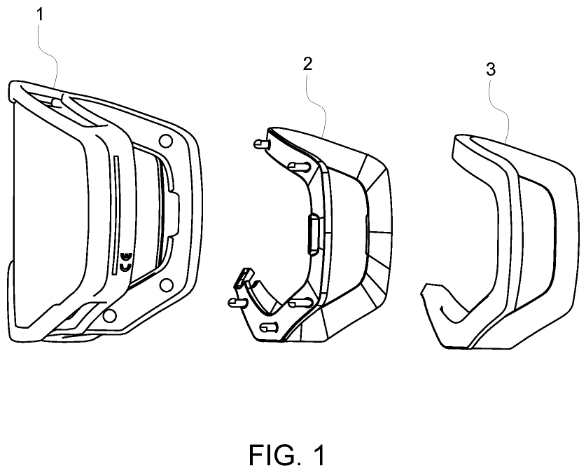

Referring to FIG. 1 to FIG. 5, the ski goggles convenient to replace include a ski goggles body 1, a mounting pad 2 and a face sponge 3, a groove 4 is formed in the inner side of the ski goggles body 1, the groove 4 is provided with two or more through holes 5, the mounting pad 2 is provided with columns 6 with the same number as the through holes 5, each column 6 penetrates through the corresponding through hole 5, and is provided with a limiting device 7 in limiting fit with the corresponding through hole 5, each limiting device 7 is positioned on one side of the corresponding column 6 and protrudes out of the side wall of the corresponding column 6, the limiting devices are small in size, so that fastening is facilitated, the cross section of a protruded portion of each limiting device 7 is semicircular, the limiting devices 7 protrude away from the center of the ski goggles body, when the face sponge 3 is replaced, the face sponge 3 is pressed with fingers to apply a force towards the palm, then each limiting device 7 moves into the range of the corresponding through hole 5, the face sponge 3 is then pulled outwards, the mounting pad 2 moves outwards to release limiting fit, replacement is facilitated, the limiting device 7 positioned on the nose part protrudes towards the center of the ski goggles body 1, when the ski goggles are used, the protruded portion of the limiting device 7 positioned on the nose part faces the inside of the ski goggles body 1, the nose of the human body provides a lifting force for the ski goggles, the limiting device positioned on the nose portion tends to move to the inside of the ski goggles body 1, thus, the limiting fit is more fastened when the ski goggles are used, and the face sponge 3 is bonded to the mounting pad 2.

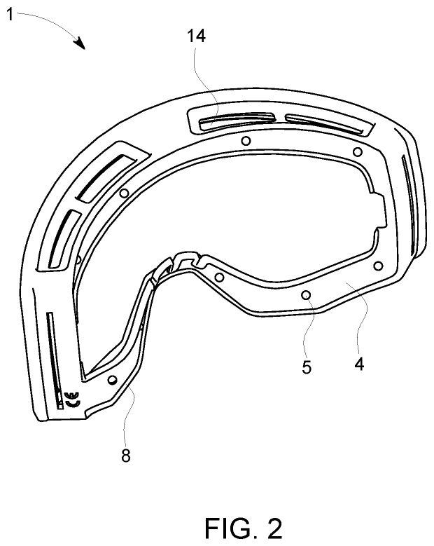

As shown in FIG. 2, the upper end face of the side wall of the groove 4 is in chamfering transition arrangement 8, so that disassembling and assembling of the mounting pad 2 are guided favorably, and then the face sponge 3 is convenient to replace.

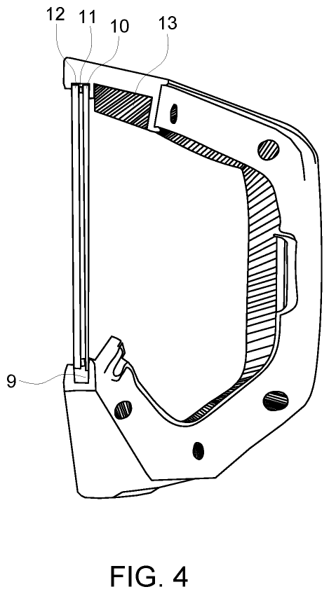

As shown in FIG. 4, a first inner cavity 9 is formed in the ski goggles body 1, a goggles lens 10, a sealing ring 11 and an anti-ultraviolet lens 12 are successively arranged in the first inner cavity 9 from the inside to the outside of the ski goggles, and the sealing ring 11 is positioned between the goggles lens 10 and the anti-ultraviolet lens 12 to prevent accessories from falling off.

As shown in FIG. 2 and FIG. 4, a second inner cavity 17 is formed in the ski goggles body 1, the second inner cavity is positioned between the first inner cavity 9 and the groove in the ski goggles body, a filling sponge 13 is arranged in the second inner cavity 17, and is closely attached to the inner wall of the ski goggles body 1, a breathable through groove 14 is formed in the outer wall of the ski goggles body 1, the filling sponge 13 stops solid matters such as snowflakes from splashing into the ski goggles, and meanwhile, gas exchange with the outside is realized in cooperation with the breathable through groove 14.

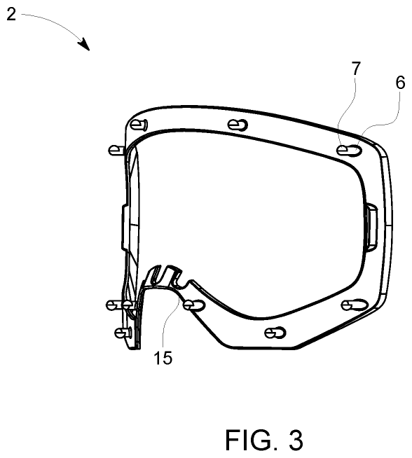

As shown in FIG. 3, two or more notches 15 are formed in the mounting pad 2 positioned on the nose part, a deformation space between the notches 15 is provided for the mounting pad 2, the mounting pad fits the structure of the nose better, the contact area with the nose is increased, then the friction force of the contact surface is increased, a buffer space is provided for deformation caused by the lifting force on the ski goggles, the ski goggles conform to the structure of the human body, the wearing comfort degree is improved, and the ski goggles are not easy to fall off.

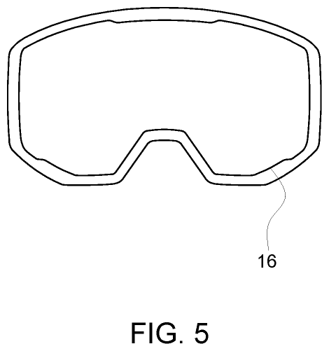

As shown in FIG. 4 and FIG. 5, limiting protruding devices 16 protruding towards the center of the first inner cavity are respectively arranged on the inner walls, located at relative positions of the nose part and located at two sides of the nose part, of the first inner cavity, and further limiting is implemented to prevent the accessories from falling off.

The foregoing embodiments are merely preferred embodiments of the present utility model, and the scope of implementation of the present utility model cannot be defined thereby. Any equivalent changes and modifications made without changing the technical concept of the present utility model are intended to be included within the scope of protection of the present utility model.

1. Ski goggles convenient to replace, comprising a ski goggles body and a mounting pad, wherein a groove is formed in the inner side of the ski goggles body, the mounting pad is mounted on the groove, the groove is provided with two or more through holes, the mounting pad is provided with columns with the same number as the through holes, a limiting device in limiting fit with the corresponding through hole is formed on each column, each limiting device is positioned on one side of the corresponding column and protrudes out of the side wall of the corresponding column, the limiting devices protrude away from the center of the ski goggles body, and the limiting device positioned on the nose part protrudes towards the center of the ski goggles body.

2. The ski goggles convenient to replace according to claim 1, wherein the upper end face of the side wall of the groove is in chamfering transition arrangement.

3. The ski goggles convenient to replace according to claim 1, wherein a first inner cavity is formed in the ski goggles body, a goggles lens, a sealing ring and an anti-ultraviolet lens are arranged in the first inner cavity from inside to outside, and the sealing ring is positioned between the goggles lens and the anti-ultraviolet lens.

4. The ski goggles convenient to replace according to claim 1, wherein a second inner cavity is formed between the first inner cavity and the groove in the ski goggles body, a filling sponge is arranged in the second inner cavity, and is closely attached to the inner wall of the second inner cavity.

5. The ski goggles convenient to replace according to claim 1, wherein two or more notches are formed in the mounting pad at the position of the nose part.

6. The ski goggles convenient to replace according to claim 3, wherein limiting protruding devices protruding towards the center of the first inner cavity are respectively arranged on the inner walls, located at relative positions of the nose part and located at two sides of the nose part, of the first inner cavity.

7. The ski goggles convenient to replace according to claim 1, wherein a face sponge is arranged on the mounting pad and is bonded to the mounting pad.

8. The ski goggles convenient to replace according to claim 1, wherein a breathable through groove is formed in the outer wall of the ski goggles body.
